(19) United States
(12) Patent Application Publication
(10) Pub. No.: US 2024/0139334 A1
(43) Pub. Date: May 2, 2024

(54) ENGINEERED LIPOSOMES FOR NEUTRALIZATION OF SARS-COV-2 AND OTHER ENVELOPED VIRUSES

(71) Applicant: Vanderbilt University, Nashville, TN (US)

(72) Inventors: Michael R. KING, Nashville, TN (US); Zhenjiang ZHANG, Nashville, TN (US)

(73) Assignee: Vanderbilt University, Nashville, TN (US)

(21) Appl. No.: 18/496,026

(22) Filed: Oct. 27, 2023

Related U.S. Application Data
(60) Provisional application No. 63/381,383, filed on Oct. 28, 2022.

(51) Int. Cl. A61K 47/69; A61P 31/14

(52) U.S. Cl. CPC A61K 47/6911; A61P 31/14

(57) ABSTRACT

The present disclosure is directed to engineered nanoparticles/liposomes that inhibit enveloped viruses and methods for use thereof.

### PRIORITY CLAIM

This application claims benefit of priority to U.S. Provisional Application Ser. No. 63/381,383, filed Oct. 28, 2022, the entire contents of which are hereby incorporated by reference.

### BACKGROUND

### 1. Field of the Disclosure

The present disclosure relates generally to the fields of medicine, infectious disease, and molecular biology. More particularly, the disclosure relates to engineered nanoparticles/liposomes that contain targeting molecules for viral attachment/entry proteins. In addition, the engineered nanoparticles/liposomes can carry reagents that, when delivered to a viral particle, have the ability to damage/destroy the viral genetic material.

### 2. Background

The 21st century has witnessed a wave of deadly viral outbreaks, none more so than the ongoing COVID-19 pandemic that is caused by severe acute respiratory syndrome coronavirus 2 (SARS-CoV-2), which has led to a dramatic loss of human life worldwide as well as devastating economic and social disruptions. As with the deadly 1918 flu outbreak a century ago, the world was unprepared for COVID-19, despite increasing dengue virus (DENV) epidemics, the 2002-2004 SARS-CoV outbreak, the 2003-2019 avian influenza epidemic, the 2009 and 2015 swine flu pandemic, the 2012 Middle East respiratory syndrome coronavirus (MERS-CoV) outbreak, the 2013-2016 Ebola virus (EBOV) epidemic, and the 2015 Zika virus (ZIKV) epidemic all causing massive morbidity and mortality with varied socioeconomic manifestations (Baker et al., 2021). Strategies for fighting against viral outbreaks include prevention (vaccines) and treatment (antiviral drugs and antibodies). In the absence of an effective vaccine at the early stages of an emerging highly pathogenic outbreak, the availability of safe and effective treatments is critically important to save lives. At present, clinically approved antiviral drugs are only available for 10 human viral pathogens, despite the vast diversity of more than 200 human viruses (Adamson et al., 2021). As viral disease emergence and re-mergence is expected to accelerate due to the massive increase in globalization and increasing human connectivity, there is a critical need to develop broad-spectrum antiviral treatments to prepare humanity for future outbreaks (Meganck & Baric, 2021).

Viruses are known to infect specific human target cells, following such sequential stages as attachment, penetration, uncoating, replication, assembly, and release (Ryu 2017). Virions are the infectious form of viruses that are highly organized nanoscale structures for the protection of the viral genome before delivering it to suitable host cells (Pellett et al., 2014). For certain viruses, the genome-containing capsid is enclosed in an envelope, which is a lipid bilayer membrane (Watanabe et al., 2019). Virion envelopes contain one or more species of virus-specified membrane glycoproteins that mediate viral entry into host cells (Cohen 2016). After entry, the viral capsid is removed and degraded by viral or host enzymes releasing the viral genomic nucleic acid. The host transcription and translation machinery is then hijacked to replicate the viral genome and proteins. Assembly of the essential viral proteins with a newly replicated viral genome yields new virions that will be released from host cells by either budding or lysis (Ryu 2017).

The viral proteins that play an essential role in the above virus replication cycle are major targets for the discovery of potent antiviral drugs (Pardi & Weissman, 2020). Approved ones include inhibitors of specific viral polymerase or proteinase, as well as specific entry/fusion inhibitors (Clercq & Li, 2016). In addition, immunomodulatory drugs have found use as indirect antiviral targets (Alijotas-Reig et al., 2020). As antivirals are not currently available for hundreds of viruses that cause human diseases, research efforts are in undeniable need to develop broadly-acting antiviral drugs as of which deadly virus will be the next to emerge into human beings is not normally predictable.

Nanomaterials have been intensively pursued in the biomedical field over the last three decades due to their uniquely appealing features for drug delivery, diagnosis, imaging and miniaturized medical devices (Patra et al., 2018). Considerable technological success has been achieved in cancer treatment, largely driven by the unmet need to reduce cancer mortality and morbidity (Shi et al., 2017). The ongoing pandemic awakened growing interest in developing nanomaterials for antiviral purposes (Tang et al., 2021). Given that the physical size of most viruses falls in the range of 20-200 nm, the relevance of engineering nanomaterials with various well-established technologies to target viruses is clear (Chakravarty & Vora, 2021). Compared with cancer cells in the complex tumor environment, viruses are much simpler in structure and function, and thus may turn out to be a relatively easy-to-conquer target. However, to date the potential for nanomaterials in directly attacking viral infections remains largely unexplored.

### SUMMARY

Thus, in accordance with the present disclosure, there is provided a method of neutralizing an enveloped virus comprising:
a) providing a nanoparticle comprising a surface exposed cell receptor molecule that mediates entry of an enveloped virus into host cells; and
b) administering the nanoparticle to an individual having or at risk of infection with the enveloped virus,
   wherein the cell receptor mediates a specific interaction event between the nanoparticle and the enveloped virus. The cell receptor molecule may be any one or a combination of two or more of the cell receptors from the following list: selectin a-Dystroglycan, transferrin, Histo-blood group antigen (HBGA) receptors, heat-shock protein 70 (HSP70), sialic acid, ephrin B2, DC-SIGN, CD4, CD21, CD81, CD155, ICAM-1, low-density lipoprotein receptor (LDLR), lactoseries tetrasaccharide c (LSTc), GM1, JAM, laminin receptor, ACE-2, L-SIGN, neclin-1/2, HVEM, nectin-4, human scavenger receptor class B, member 2 (SCARB2), P-selectin glycoprotein ligand-1 (PSGL 1), glucose transporter type 1 (GLUT-1), neuropilin-1, coxsackievirus adenovirus receptor (CAR), av integrins, T-cell immunoglobulin and mucin domain 1 (TIM-1), human Niemann-Pick C1 (NPC1), scavenger receptor class B type I (SR-B1), major histocompatibility complex class II (MHC-11), decay-accelerating factor (OAF), CAR (occluding), integrins, and/or C-C chemokine receptor type 5 (CCR5).

The enveloped virus may be coronavirus (CoV), such as SARS-CoV-2, Norovirus, Japanese encephalitis virus, Influenza A, Henipahvirus, Bunyavirus, Hepatitis A virus, Poliovirus, Rhinovirus (major group), Rhinovirus (minor group), John Cunningham polyomavirus, SV40 polyomavirus, Reovirus, Sindbis virus, Herpes simplex virus 1/2, Measles virus, Enterovirus 71, Human T cell leukemia virus 1, Adenovirus 2, Ebola virus, hepatitis C virus, Epstein-Barr virus, Rotavirus, or HIV.

The nanoparticle may further comprise a DNA or RNA degrading reagent, such as any one or any combination from the following list: DNase I, DNase II, micrococcal nuclease, RNase A, RNase D, RNase E, RNase G, RNase H, RNase L, RNase P, RNase PH, RNase PhyM, RNase R, RNase T, RNase T1, RNase T2, RNase V, RNase U2, Oligoribonuclease, Exoribonuclease I, and/or Exoribonuclease II.

The nanoparticle may be a liposome, such as unilamellar liposomes, wherein majority of the liposomes have a size from 30 to 500 nm. Administration may comprise systemic administration, regional administration, or administration local to a site of infection, such as intravenous, intra-arterials, intranasal, inhalation, oral, parenteral, vaginal, or rectal. The nanoparticles may be administered into the circulation of an individual by intravenous administration. The nanoparticles may be administered into the lymphatic circulation of the individual. The method may further comprise treating the individual with a second anti-viral therapy.

Also provided is a nanoparticle comprising a surface exposed cell receptor molecule that mediates entry of an enveloped virus into host cells. The cell receptor molecule may be any one or any combination of two or more cell receptors from the following list: selectin a-Dystroglycan, transferrin, Histo-blood group antigen (HBGA) receptors, heat-shock protein 70 (HSP70), sialic acid, ephrin B2, DC-SIGN, CD4, CD21, CD81, CD155, ICAM-1, low-density lipoprotein receptor (LDLR), lactoseries tetrasaccharide c (LSTc), GM1, JAM, laminin receptor, ACE-2, L-SIGN, neclin-1/2, HVEM, nectin-4, human scavenger receptor class B, member 2 (SCARB2), P-selectin glycoprotein ligand-1 (PSGL 1), glucose transporter type 1 (GLUT-1), neuropilin-1, coxsackievirus adenovirus receptor (CAR), av integrins, T-cell immunoglobulin and mucin domain 1 (TIM-1), human Niemann-Pick C1 (NPC1), scavenger receptor class B type I (SR-B1), major histocompatibility complex class II (MHC-11), decay-accelerating factor (OAF), CAR (occluding), integrins, and/or C-C chemokine receptor type 5 (CCR5). The nanoparticle may further comprise a DNA or RNA degrading reagent, such as any one of any combination from the following list: DNase I, DNase II, micrococcal nuclease, RNase A, RNase D, RNase E, RNase G, RNase H, RNase L, RNase P, RNase PH, RNase PhyM, RNase R, RNase T, RNase T1, RNase T2, RNase V, RNase U2, Oligoribonuclease, Exoribonuclease I, and/or Exoribonuclease II. The nanoparticle may be a liposome, such as unilamellar liposomes, wherein majority of the liposomes have a size from 30 to 250 nm.

Also provided is a kit comprising a nanoparticle comprising a surface exposed cell receptor molecule that mediates entry of an enveloped virus into host cells. The cell receptor molecule may be any one or any combination of two or more cell receptors from the following list: selectin a-Dystroglycan, transferrin, Histo-blood group antigen (HBGA) receptors, heat-shock protein 70 (HSP70), sialic acid, ephrin B2, DC-SIGN, CD4, CD21, CD81, CD155, ICAM-1, low-density lipoprotein receptor (LDLR), lactoseries tetrasaccharide c (LSTc), GM1, JAM, laminin receptor, ACE-2, L-SIGN, neclin-1/2, HVEM, nectin-4, human scavenger receptor class B, member 2 (SCARB2), P-selectin glycoprotein ligand-1 (PSGL 1), glucose transporter type 1 (GLUT-1), neuropilin-1, coxsackievirus adenovirus receptor (CAR), av integrins, T-cell immunoglobulin and mucin domain 1 (TIM-1), human Niemann-Pick C1 (NPC1), scavenger receptor class B type I (SR-B1), major histocompatibility complex class II (MHC-11), decay-accelerating factor (OAF), CAR (occluding), integrins, and/or C-C chemokine receptor type 5 (CCR5). The nanoparticle may further comprise a DNA or RNA degrading reagent, such as any one of any combination from the following list: DNase I, DNase II, micrococcal nuclease, RNase A, RNase D, RNase E, RNase G, RNase H, RNase L, RNase P, RNase PH, RNase PhyM, RNase R, RNase T, RNase T1, RNase T2, RNase V, RNase U2, Oligoribonuclease, Exoribonuclease I, and/or Exoribonuclease II. The nanoparticle may be a liposome, such as unilamellar liposomes, wherein majority of the liposomes have a size from 30 to 250 nm.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-E. Characterization of liposomes. (FIG. 1A) Size distribution of naked and rhACE2-conjugated liposomes as measured by dynamic light scattering (DLS). Visualization of naked liposomes (FIG. 1B) and rhACE2-conjugated liposomes (FIG. 1C) by TEM with negative staining. The white arrows denote rhACE2 proteins. Scale bar=100 nm. (FIG. 1D) Confirmation of the association of rhA-CE2 on the liposome surface using liposomes without NTA-Ni²⁺ as a control. **P<0.01. (FIG. 1E) Colloidal stability of liposomes stored at 4° C. monitored by size measurement with DLS.

FIGS. 2A-F. Construction and characterization of SARS-CoV-2 spike pseudotyped lentivirus. (FIG. 2A) SARS-CoV-2 pseudotyping in 293T cells. Green is GFP. Scale bar=50 μm. (FIG. 2B) Visualization of SARS-CoV-2 pseudovirions by TEM with negative staining. (FIG. 2C) Enlarged TEM image to show the spike proteins on the surface. (FIG. 2D) Spike protein dissociation from the membrane of pseudovirus after purification. Spikes are partly (FIG. 2E) or completely (FIG. 2F) dissociated from the pseudovirus. White arrows denote spike proteins that remain attached to the membrane of the pseudovirus. In all the TEM images, scale bar=100 nm.

FIGS. 3A-B. Pseudovirus infectivity to ACE2 and TMPRSS2 expressing 293T cells. (FIG. 3A) Fluorescent images of 293T cells infected with GFP labelled pseudovirus at the specified dilution factors. Scale bar=50 μm. (FIG. 3B) Quantified infectivity of 293T cells with luciferase labeled pseudo-virus. Pseudovirus infectivity was expressed in terms of relative luminescence unit (RLU).

FIGS. 4A-D. Neutralization of SARS-CoV-2 pseudovirus by rhACE2 liposomes. (FIG. 4A) Infection of ACE2 and TMPRSS2 expressing 293T cells. (FIGS. 4B-D) TEM images of pseudovirus incubated with naked liposomes (FIG. 4B) and rhACE2 liposomes at higher (FIG. 4C) and lower (FIG. 4D) magnification. Black and white arrows denote liposomes and pseudovirus, respectively. White arrow heads denote spike proteins. In all the TEM images, scale bar=100 nm.

### DESCRIPTION OF ILLUSTRATIVE EMBODIMENTS

The devastating COVID-19 pandemic, along with many other recent viral outbreaks around the world, defines the urgent need for the development of safe and effective antivirals to reduce morbidity and mortality associated with infection. SARS-CoV-2 attaches to host cells via the same angiotensin-converting enzyme 2 (ACE2) receptor as SARS-CoV using its spike glycoprotein (Hoffmann et al., 2020). Upon binding, the spike protein is primed by the transmembrane protease serine 2 (TMPRSS2) on the cell surface to initiate cellular entry. Several studies have shown that recombinant human ACE2 (rhACE2) and antibodies against the spike protein both blocked cellular entry of the virus in vitro (Monteil et al., 2020; Barnes et al., 2020; Li et al., 2022). Inspired by these findings, and compelled by the potential of nanotechnology to combat viral outbreaks, the inventors developed nanoscale liposomes that are coated with this cellular receptor for SARS-CoV-2). Lentiviral particles pseudotyped with the spike protein of SARS-CoV-2 are constructed and used to test the virus neutralization potential of the engineered liposomes. Under TEM, the inventors observed dissociation of spike proteins from the pseudovirus surface for the first time. In addition, the liposomes potently inhibit viral entry into host cells by extracting the spike proteins from the pseudovirus surface. As the receptor on the liposome surface can be readily substituted to target other viruses, the receptor-coated liposome represents a promising strategy for broad spectrum antiviral development.

These and other aspects of the disclosure are described in detail below.

### I. ENVELOPED VIRUSES

A virus is a submicroscopic infectious agent that replicates only inside the living cells of an organism. Viruses infect all life forms, from animals and plants to microorganisms, including bacteria and archaea. More than 9,000 virus species have been described in detail, which is like a fraction of the millions of types of viruses that exist. Viruses are found in almost every ecosystem on Earth and are the most numerous type of biological entity.

When infected, a host cell is often forced to rapidly produce thousands of copies of the original virus. When not inside an infected cell or in the process of infecting a cell, viruses exist in the form of independent particles, or virions, consisting of (i) the genetic material, i.e., long molecules of DNA or RNA that encode the structure of the proteins by which the virus acts; (ii) a protein coat, the capsid, which surrounds and protects the genetic material; and in some cases (iii) an outside envelope of lipids. The shapes of these virus particles range from simple helical and icosahedral forms to more complex structures. Most virus species have virions too small to be seen with an optical microscope and are one-hundredth the size of most bacteria.

Species of viruses that envelop themselves in a modified form of one of the cell membranes are called enveloped viruses. The membrane may be derived from either the outer membrane surrounding an infected host cell or internal membranes, such as a nuclear membrane or endoplasmic reticulum. This membrane is studded with proteins coded for by the viral genome and host genome; the lipid membrane itself and any carbohydrates present originate entirely from the host. Influenza virus, HIV (which causes AIDS), and severe acute respiratory syndrome coronavirus 2 (which causes COVID-19) use this strategy. Most enveloped viruses are dependent on the envelope for their infectivity.

A viral envelope is the outermost layer of many types of viruses. It protects the genetic material in their life cycle when traveling between host cells. They may also help viruses avoid the host immune system. Glycoproteins on the surface of the envelope serve to identify and bind to receptor sites on the host's membrane. The viral envelope then fuses with the host's membrane, allowing the capsid and viral genome to enter and infect the host.

The cell from which a virus buds often dies or is weakened, and sheds more viral particles for an extended period. The lipid bilayer envelope of these viruses is relatively sensitive to desiccation, heat, and amphiphiles such as soap and detergents, therefore these viruses are easier to sterilize than non-enveloped viruses, have limited survival outside host environments, and typically must transfer directly from host to host. Enveloped viruses possess great adaptability and can change in a short time in order to evade the immune system. Enveloped viruses can cause persistent infections.

Enveloped DNA viruses include Herpesviridae, Poxviridae, Hepadnaviridae, Baculoviridae, Fuselloviridae, Iridoviridae, Lipothrixviridae, Plasmaviridae, Polydnaviridae and Asfarviridae. Enveloped RNA viruses include Bunyaviridae, Coronaviridae, Filoviridae, Flaviviridae, Hepatitis D viridae, Hypoviridae, Orthomyxoviridae, Paramyxoviridae, Pneumoviridae, Retroviridae, Rhabdoviridae, Togaviridae, Arenaviridae, Arteriviridae, and Cystoviridae. All those underlined are known to us humans as a natural host or can be disease causing in humans.

### II. NANOPARTICLES

As used herein, the term “nanoparticle” refers to any material having dimensions in the 1-1,000 nm range. In some embodiments, nanoparticles have dimensions in the 50-500 nm range. Nanomaterials can be categorized into four types such as: (1) inorganic-based nanomaterials; (2) carbon-based nanomaterials; (3) organic-based nanomaterials; and (4) composite-based nanomaterials.

Generally, inorganic-based nanomaterials include different metal and metal oxide nanomaterials. Examples of metal-based inorganic nanomaterials are silver (Ag), gold (Au), aluminum (Al), cadmium (Cd), copper (Cu), iron (Fe), zinc (Zn), and lead (Pb) nanomaterials, whereas examples of metal oxide-based inorganic nanomaterials are zinc oxide (ZnO), copper oxide (CuO), magnesium aluminum oxide (MgAl₂O₄), titanium dioxide (TiO₂), cerium oxide (CeO₂), iron oxide (Fe₂O₃), silica (SiO₂), and iron oxide (Fe₃O₄), etc. Carbon-based nanomaterials include graphene, fullerene, single-walled carbon nanotube, multiwalled carbon nanotube, carbon fiber, an activated carbon, and carbon black. The organic-based nanomaterials are formed from organic materials excluding carbon materials, for instance, dendrimers, cyclodextrin, liposome, and micelle. The composite nanomaterials are any combination of metal-based, metal oxide-based, carbon-based, and/or organic-based nanomaterials, and these nanomaterials have complicated structures like a metal-organic framework.”

Nanoparticles include such nanoscale materials as a lipid-based nanoparticle, a superparamagnetic nanoparticle, a nanoshell, a semiconductor nanocrystal, a quantum dot, a polymer-based nanoparticle, a silicon-based nanoparticle, a silica-based nanoparticle, a metal-based nanoparticle, a fullerene and a nanotube (Ferrari, 2005). The conjugation of polypeptide or nucleic acids to nanoparticles provides structures with potential application for targeted delivery, controlled release, enhanced cellular uptake and intracellular trafficking, and molecular imaging of therapeutic peptides in vitro and in vivo (West, 2004; Stayton et al., 2000; Ballou et al., 2004; Frangioni, 2003; Dubertret et al., 2002; Michalet et al., 2005; Dwarakanath et al., 2004).

### A. Lipid-Based Nanoparticles

Lipid-based nanoparticles include liposomes, solid lipid nanoparticles and nanostructured lipid carriers. Lipid-based nanoparticles may be positively charged, negatively charged or neutral. In certain embodiments, the lipid-based nanoparticle is neutrally charged (e.g., a DOPC liposome). Liposome size can vary from very small (0.025 μm) to large (2.5 μm) vesicles. Moreover, liposomes may have one or multiple lipid bilayer membranes. Thus, liposomes can be classified as multilamellar vesicles (MLV) or unilamellar vesicles, the latter being further classified as large unilamellar vesicles (LUV) or small unilamellar vesicles (SUV). In unilamellar liposomes, the vesicle has a single phospholipid bilayer sphere enclosing the aqueous solution. In multilamellar liposomes, vesicles have an onion structure. Several unilamellar vesicles can form on the inside of the other with smaller size, making a multilamellar structure of concentric phospholipid spheres separated by layers of water.

A “liposome” is a generic term encompassing a variety of single or multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition. Liposomes provided herein include unilamellar liposomes, multilamellar liposomes and multivesicular liposomes. Liposomes provided herein may be positively charged, negatively charged or neutrally charged.

A multilamellar liposome has multiple lipid layers separated by aqueous medium. They form spontaneously when lipids comprising phospholipids are suspended in an excess of aqueous solution. The lipid components undergo self-rearrangement before the formation of closed structures and entrap water and dissolved solutes between the lipid bilayers (Ghosh & Bachhawat, 1991). Lipophilic molecules or molecules with lipophilic regions may also dissolve in or associate with the lipid bilayer.

In specific aspects, materials may be encapsulated in the aqueous interior of a liposome, interspersed within the lipid bilayer of a liposome, attached to a liposome via a linking molecule that is associated with the liposome, complexed with the liposome, or the like.

The methods of preparing the liposomes usually involve four basic stages: drying down lipids from organic solvent, dispersing the lipid in aqueous media, purifying the resultant liposome and analyzing the final product. A liposome used according to the present disclosure can be made by different methods, as would be known to one of ordinary skill in the art. For example, a phospholipid is dissolved in a solvent, such as tert-butanol. The lipid(s) can then be mixed with an agent to be encapsulated or incorporated. Tween 20 is added to the lipid mixture such that Tween 20 is about 5% of the composition's weight. Excess solvent is added to this mixture such that the volume of solvent is at least 95%. The mixture is vortexed, frozen in a dry ice/acetone bath and lyophilized overnight. The lyophilized preparation is stored at −20° C. and can be used up to three months. When required the lyophilized liposomes are reconstituted in 0.9% saline.

Alternatively, a liposome can be prepared by mixing lipids in a solvent in a container, e.g., a glass, pear-shaped flask. Using a rotary evaporator, the solvent is removed at approximately 40° C. under negative pressure. The solvent normally is removed within about 5 min. to 2 hours, depending on the desired volume of the liposomes. The composition can be dried further in a desiccator under vacuum.

Liposomes may be loaded with drugs using either passive (during formation) or active (after formation) methods. Passive methods include mechanical dispersion, solvent dispersion and detergent removal (of non-encapsulated materials). A variety of mechanical dispersion methods exist including sonication, French pressure cell/extrusion, freeze-thawing, lipid film hydration, micro-emulsification, membrane extrusion and dried reconstitution.

Dried lipids can be hydrated at approximately 0.1-50 mM phospholipid in sterile, pyrogen-free water by shaking until all the lipid film is resuspended. The aqueous liposomes can be then separated into aliquots, each placed in a vial, lyophilized and sealed under vacuum.

The dried liposomes or lyophilized liposomes prepared as described above may be reconstituted in a solution of a protein or peptide and diluted to an appropriate concentration with a suitable solvent, e.g., DPBS. The mixture is then vigorously shaken in a vortex mixer. Unencapsulated additional materials, such as agents including but not limited to hormones, drugs, nucleic acid constructs and the like, are removed by centrifugation at 29,000×g and the liposomal pellets washed. The washed liposomes are resuspended at an appropriate total phospholipid concentration, e.g., about 50-200 mM. The amount of additional material or active agent encapsulated can be determined in accordance with standard methods. After determination of the amount of additional material or active agent encapsulated in the liposome preparation, the liposomes may be diluted to appropriate concentrations and stored at 4° C. until use. A pharmaceutical composition comprising the liposomes will usually include a sterile, pharmaceutically acceptable carrier or diluent, such as water or saline solution.

In other alternative methods, liposomes can be prepared in accordance with other known laboratory procedures (e.g., see Bangham et al., 1965; Gregoriadis, 1979; Deamer and Uster, 1983; Szoka and Papahadjopoulos, 1978, each incorporated herein by reference in relevant part). Additional liposomes which may be useful with the present embodiments include cationic liposomes, for example, as described in WO02/100435A1, U.S. Pat. No. 5,962,016, U.S. Application 2004/0208921, WO03/015757A1, WO04029213A2, U.S. Pat. Nos. 5,030,453, and 6,680,068, all of which are hereby incorporated by reference in their entirety without disclaimer. A process of making liposomes is also described in WO04/002453A1. Neutral lipids can be incorporated into cationic liposomes (e.g., Farhood et al., 1995). Various neutral liposomes which may be used in certain embodiments are disclosed in U.S. Pat. No. 5,855,911, which is incorporated herein by reference. These methods differ in their respective abilities to entrap aqueous material and their respective aqueous space-to-lipid ratios.

The size of a liposome varies depending on the method of synthesis. Liposomes in the present embodiments can be a variety of sizes. In certain embodiments, the liposomes are small, e.g., less than about 100 nm, about 90 nm, about 80 nm, about 70 nm, about 60 nm, or less than about 50 nm in external diameter. For example, in general, prior to the incorporation of nucleic acid, a liposome for use according to the present embodiments comprises a size of about 50 to 500 nm. Such liposome formulations may also be defined by particle charge (zeta potential) and/or optical density (OD). For instance, a liposome formulation will typically comprise an OD₄₀₀ of less than 0.45 prior to nucleic acid incorporation. Likewise, the overall charge of such particles in solution can be defined by a zeta potential of about 50-80 mV.

In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040; U.K. Patent Application GB 2193095 A; Mayer et al., 1986; Hope et al., 1985; Mayhew et al. 1987; Mayhew et al., 1984; Cheng et al., 1987; and Liposome Technology, 1984, each incorporated herein by reference).

In certain embodiments, the lipid-based nanoparticle is a neutral liposome (e.g., a DOPC liposome). “Neutral liposomes” or “non-charged liposomes”, as used herein, are defined as liposomes having one or more lipid components that yield an essentially neutral, net charge (substantially non-charged). By “essentially neutral” or “essentially non-charged”, it is meant that few, if any, lipid components within a given population (e.g., a population of liposomes) include a charge that is not canceled by an opposite charge of another component (i.e., fewer than 10% of components include a non-canceled charge, more preferably fewer than 5%, and most preferably fewer than 1%). In certain embodiments, neutral liposomes may include mostly lipids and/or phospholipids that are themselves neutral under physiological conditions (i.e., at about pH 7).

Liposomes and/or lipid-based nanoparticles of the present embodiments may comprise a phospholipid. In certain embodiments, a single kind of phospholipid may be used in the creation of liposomes (e.g., a neutral phospholipid, may be used to generate neutral liposomes). In other embodiments, more than one kind of phospholipid may be used to create liposomes.

Phospholipids include, for example, phosphatidylcholines, phosphatidylglycerols, and phosphatidylethanolamines; because phosphatidylethanolamines and phosphatidyl cholines are non-charged under physiological conditions (i.e., at about pH 7), these compounds may be particularly useful for generating neutral liposomes. In certain embodiments, the phospholipid DOPC is used to produce non-charged liposomes. In certain embodiments, a lipid that is not a phospholipid (e.g., a cholesterol) may be used.

Phospholipids include glycerophospholipids and certain sphingolipids. Phospholipids include, but are not limited to, dioleoylphosphatidylycholine (“DOPC”), egg phosphatidylcholine (“EPC”), dilauryloylphosphatidylcholine (“DLPC”), dimyristoylphosphatidylcholine (“DMPC”), dipalmitoylphosphatidylcholine (“DPPC”), distearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dilauryloylphosphatidylglycerol (“DLPG”), dimyristoylphosphatidylglycerol (“DMPG”), dipalmitoylphosphatidylglycerol (“DPPG”), distearoylphosphatidylglycerol (“DSPG”), distearoyl sphingomyelin (“DSSP”), distearoylphophatidylethanolamine (“DSPE”), dioleoylphosphatidylglycerol (“DOPG”), dimyristoyl phosphatidic acid (“DMPA”), dipalmitoyl phosphatidic acid (“DPPA”), dimyristoyl phosphatidylethanolamine (“DMPE”), dipalmitoyl phosphatidylethanolamine (“DPPE”), dimyristoyl phosphatidylserine (“DMPS”), dipalmitoyl phosphatidylserine (“DPPS”), brain phosphatidylserine (“BPS”), brain sphingomyelin (“BSP”), dipalmitoyl sphingomyelin (“DPSP”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), dioleoylphosphatidylethanolamine (“DOPE”), palmitoyloeoyl phosphatidylcholine (“POPC”), palmitoyloeoyl phosphatidylethanolamine (“POPE”), lysophosphatidylcholine, lysophosphatidylethanolamine, and dilinoleoylphosphatidylcholine.

Phospholipids may be from natural or synthetic sources. However, phospholipids from natural sources, such as egg or soybean phosphatidylcholine (e.g., hydro Soy PC lipids), brain phosphatidic acid, brain or plant phosphatidylinositol, heart cardiolipin and plant or bacterial phosphatidylethanolamine are not used, in certain embodiments, as the primary phosphatide (i.e., constituting 50% or more of the total phosphatide composition) because this may result in instability and leakiness of the resulting liposomes.

### B. Purification

In certain embodiments, the nanoparticles/liposomes of the present disclosure may be purified. The term “purified,” as used herein, is intended to refer to a composition, isolatable from other components, wherein the material is purified to any degree relative to its starting state. Where the term “substantially purified” is used, this designation will refer to a composition in which the material forms the major component of the composition, such as constituting about 50%, about 60%, about 70%, about 80%, about 90%, about 95% or more of the composition.

Purification techniques are well known to those of skill in the art. Purification of nanoparticles/liposomes includes separating or removing non-encapsulated materials as well as detergent used in the preparation process. This is important for quality control of liposomal products. Some methods for liposome purification include dialysis, column chromatographic separation methods, centrifugation, protamine aggregation methods, ion-exchange resin, and ultrafiltration methods. To achieve effective purification, suitable methods should be chosen based on the characteristics of every individual liposome and optimization of separation condition is also demanded. Various methods for quantifying the degree of purification are also known to those of skill in the art in light of the present disclosure.

### C. Introduction of Targeting Agents

Post-functionalization of nanoparticles/liposomes with a variety of agents, including targeting ligands, by direct conjugation to the surface of preformed liposomes has been extensively explored. However, the reaction conditions for attaching the targeting ligand may cause destabilization of the liposomes. In addition, attachment of large targeting molecules may decrease targeting efficiency due to conformational changes. In addition, the targeting molecule may alter the stability of the liposomes. This problem may be avoided by using longer linking moieties.

A variety of different methods for coupling targeting ligands to the surface of preformed liposomes include traditional approaches such as amine-functionalization, carboxylic acid-functionalization, para-Nitrophenylcarbonyl-functionalization, thiol-functionalization, and maleimide-functionalization, and more recent approaches such as copper(I)-catalyzed Huisgen 1,3-dipolar cycloaddition, copper-free click chemistry, Staudinger ligation, tetrazine/trans-cyclooctene inverse electron demand Diels-Alder cycloaddition, enzymatic modification, and His-tag chelating strategy.

### III. ANTI-VIRAL PAYLOAD

In certain embodiments, it is envisioned that the nanoparticles/liposomes will contain a payload material that can be delivered to a viral particle. In particular, it is envisioned that one, two or more DNA and/or RNA degrading reagents may be included. Examples of such agents include DNase I, DNase II, micrococcal nuclease, RNase A, RNase D, RNase E, RNase G, RNase H, RNase L, RNase P, RNase PH, RNase PhyM, RNase R, RNase T, RNase T1, RNase T2, RNase V, RNase U2, Oligoribonuclease, Exoribonuclease I, and/or Exoribonuclease II.

### IV. FORMULATIONS AND METHODS OF TREATING INFECTIOUS DISEASES

The present disclosure envisions administration of pharmaceutical formulations comprising nanoparticles/liposomes as described herein. Therapeutically effective amounts of these materials can be administered by a number of routes, including parenteral administration, for example, intravenous, intraperitoneal, intramuscular, intraarticular injection, or infusion. In particular aspects, the materials are administered directly to a site infection or systemically. The therapeutically effective amount of the materials is that amount that achieves a desired effect in a subject being treated. Such an effect includes reducing or eliminating any symptom of viral infect, reducing viral load and/or replication, inhibiting viral infection, or limiting the course of viral infection.

The nanoparticles/liposomes can be administered in treatment regimens consistent with the disease, for example a single or a few doses over one to several days to ameliorate an infection or periodic doses over an extended time to inhibit disease progression and prevent disease recurrence. The precise dose to be employed in the formulation will also depend on the route of administration, and the seriousness of the infection, and should be decided according to the judgment of the practitioner and each patient's circumstances. The therapeutically effective amount of nanoparticles/liposomes will be dependent on the subject being treated, the severity and type of the infection, and the manner of administration. The exact amount of nanoparticles/liposomes is readily determined by one of skill in the art based on the age, weight, sex, and physiological condition of the subject. Effective doses can be extrapolated from dose-response curves derived from in vitro or animal model test systems.

### A. Pharmaceutical Compositions

Certain of the methods set forth herein pertain to methods involving the administration of a pharmaceutically effective amount of a composition comprising nanoparticles/liposomes as described in the present disclosure. For delivery to a subject, the nanoparticles/liposomes will be dispersed in a pharmaceutically acceptable carrier, diluent or buffer.

As used herein, “pharmaceutically acceptable carrier or diluent” includes any and all solvents, dispersion media, coatings, surfactants, antioxidants, preservatives (e.g. , antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (Remington's, 1990). Except insofar as any conventional carrier is incompatible with the active ingredient, its use in the therapeutic or pharmaceutical compositions is contemplated. The compositions used in the present disclosure may comprise different types of carriers depending on whether it is to be administered in solid, liquid or aerosol form, and whether it need to be sterile for such routes of administration as injection.

The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions, and these are discussed in greater detail below. For human administration, preparations preferably meet sterility, pyrogenicity, general safety and purity standards as required by FDA Office of Biologics standards.

The present disclosure contemplates methods using compositions that are sterile solutions for injection or for application by any other route as discussed in greater detail below. A person of ordinary skill in the art would be familiar with techniques for generating sterile solutions for injection or application by any other route. Sterile injectable solutions are prepared by incorporating the active compounds in the required amount in the appropriate solvent with various of the other ingredients familiar to a person of skill in the art.

The formulation of the composition may vary depending upon the route of administration. For parenteral administration in an aqueous solution, for example, the solution should be suitably buffered and the liquid diluent first rendered isotonic with sufficient saline or glucose. In this connection, sterile aqueous media which can be employed will be known to those of skill in the art in light of the present disclosure.

In addition to the compounds formulated for parenteral administration, such as intravenous or intramuscular injection, other pharmaceutically acceptable forms include formulations for administration via an implantable drug delivery device, and any other form. One may also use nasal solutions or sprays, aerosols or inhalants in the present disclosure.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. A person of ordinary skill in the art would be familiar with well-known techniques for preparation of oral formulations.

In certain embodiments, pharmaceutical composition includes at least about 0.1% by weight of the active agent. The composition may include, for example, about 0.01%. In other embodiments, the pharmaceutical composition includes about 2% to about 75% of the weight of the composition, or between about 25% to about 60% by weight of the composition, for example, and any range derivable therein.

The pharmaceutical composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof. The composition may be stable under the conditions of manufacture and storage, and preserved against the contaminating action of microorganisms, such as bacteria and fungi. It will be appreciated that exotoxin contamination should be kept minimally at a safe level, for example, less than 0.5 ng/mg protein.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. In many cases, it will be preferable to include isotonic agents, such as, for example, sugars, sodium chloride or combinations thereof.

In other embodiments, one may use nasal solutions or sprays, aerosols or inhalants in the present disclosure. Nasal solutions may be aqueous solutions designed to be administered to the nasal passages in drops or sprays.

Sterile injectable solutions are prepared by incorporating the nanoparticles in the required amount in the appropriate solvent with various of the other ingredients enumerated above, as required, followed by sterilization.

### B. Dosage

A pharmaceutically effective amount of the nanoparticles/liposomes is determined based on the intended goal, for example, treating one or more symptoms of a viral infection. The quantity to be administered, both according to number of treatments and dose, depends on the subject to be treated, the state of the subject, the protection desired, and the route of administration. Precise amounts of the nanoparticles/liposomes also depend on the judgment of the practitioner and are peculiar to each individual.

For example, a dose of the nanoparticles/liposomes may be about 0.0001 milligrams to about 1.0 milligrams, or about 0.001 milligrams to about 0.1 milligrams, or about 0.1 milligrams to about 1.0 milligrams, or even about 10 milligrams per dose or so. Multiple doses can also be administered. In some embodiments, a dose is at least about 0.0001 milligrams. In further embodiments, a dose is at least about 0.001 milligrams. In still further embodiments, a dose is at least 0.01 milligrams. In still further embodiments, a dose is at least about 0.1 milligrams. In more particular embodiments, a dose may be at least 1.0 milligrams. In even more particular embodiments, a dose may be at least 10 milligrams. In further embodiments, a dose is at least 100 milligrams or higher.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

The dose can be repeated as determined by those of ordinary skill in the art. Thus, in some embodiments of the methods set forth herein, a single dose is contemplated. In other embodiments, two or more doses are contemplated. Where more than one dose is administered to a subject, the time interval between doses can be any time interval as determined by those of ordinary skill in the art. For example, the time interval between doses may be about 1 hour to about 2 hours, about 2 hours to about 6 hours, about 6 hours to about 10 hours, about 10 hours to about 24 hours, about 1 day to about 2 days, about 1 week to about 2 weeks, or longer, or any time interval derivable within any of these recited ranges.

In certain embodiments, the method may provide a continuous supply of a pharmaceutical composition to the patient. This could be accomplished by catheterization, followed by continuous administration of the therapeutic agent. The administration could be intra-operative or post-operative.

### C. Combination Treatments

Certain embodiments of the present disclosure provide for the administration or application of one or more secondary forms of therapies for the treatment or prevention of a viral infection. The secondary form of therapy may be administration of one or more secondary pharmacological agents that can be applied in the treatment or prevention of viral infection. If the secondary therapy is a pharmacological agent, it may be administered prior to, concurrently, or following administration of the nanoparticles/liposomes.

The interval between the administration of the nanoparticles/liposomes and the secondary therapy may be any interval as determined by those of ordinary skill in the art. For example, the interval may be minutes to weeks. In embodiments where the agents are separately administered, one would generally ensure that a long period of time did not expire between the time of each delivery, such that each therapeutic agent would still be able to exert an advantageously combined effect on the subject. For example, the interval between therapeutic agents may be about 12 h to about 24 h of each other and, more preferably, within about 6 hours to about 12 h of each other. In some situations, the time period for treatment may be extended, however, where several days (2, 3, 4, 5, 6 or 7) to several weeks (1, 2, 3, 4, 5, 6, 7 or 8) lapse between the respective administrations. In some embodiments, the timing of administration of a secondary therapeutic agent is determined based on the response of the subject to the nanoparticles.

Various combinations may be employed. For the example below a nanoparticle/liposome as described herein is “A” and another anti-viral therapy is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

### V. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Cells and Materials. Regular 293T cells were purchased from ATCC (CRL-3216) and maintained in high glucose DMEM (GIBCO) supplemented with 10% FBS, penicillin (100 IU/ml), streptomycin (100 μg/ml) and L-Glutamine at 37° C. 293T cells that stably express human ACE2 and TMPRSS2 were purchased from InvivoGen (hkb-hace2) and maintained and used following the manufacturer's instructions. The lipids for liposome preparation, including hydrogenated soy phosphatidylcholine (HSPC), cholesterol, 18:1 DGS-NTA(Ni) and 18:0 PEG2000 PE (DSPE-PEG) were purchased from Avanti Polar Lipids. rhACE2 protein with his-tag was purchased from Sino Biological (10108-H08H). Plasmids for SARS-CoV-2 pseudovirus construction, including a mixture of two plasmids, one for lentiviral packaging the other encoding SARS-CoV-2 spike, and two reporter vector pLVXS-ZsGreen1-Puro and pLVXS-Luciferase-Puro, and Lenti-X Concentrator was purchased from Takara Bio. The reported vectors were amplified in E. coli and stored at 4° C. before use. Luciferase Assay System was purchased from Promega (E4030).

Preparation and Characterization of Liposomes. Liposomes were prepared using an extrusion method using Mini-Extruder (Avanti Polar Lipids). First, a lipid film was prepared with a molar composition of HSPC:cholesterol:DGS-NTA(Ni):DSPE-PEG=56%:38%:4%:2%. The lipid film was then hydrated using PBS buffer at 65° C. for 1 h before the mixture was extruded through a 100 nm polycarbonate membrane 10 times. To prepare rhACE2-cojugated liposomes, the protein was incubated with preformed liposomes at 37° C. for 1 h. The size distribution of liposomes was measured by dynamic light scattering (DLS) using a Zetasizer (Marvin Panalytical). The liposomes were also imaged using TEM with negative staining (1% buffered phosphotungstic acid) to analyze the morphology.

Construction of Pseudovirus. Pseudoviruses bearing the spike protein of SARS-CoV-2 and carrying either green fluorescent protein (GFP) or a firefly luciferase reporter gene were produced in human embryonic kidney 293T cells using packaging plasmids obtained from Takara Bio following the manufacturer's instructions. Plasmids for SARS-CoV-2 pseudovirus construction, including a mixture of two plasmids, one for lentiviral packaging the other encoding SARS-CoV-2 spike, and two reporter vectors, pLVXS-ZsGreen1-Puro and pLVXS-Luciferase-Puro, and Lenti-X Concentrator, were purchased from Takara Bio. These vectors were amplified in E. coli and stored at 4° C. before use. Pseudovirus was imaged using TEM with negative staining to analyze the morphology. 293T cells expressing both ACE2 and TMPRSS2 was used to analyze the infectivity of pseudovirus.

Pseudovirus Neutralization. Neutralization was measured by the reduction in luciferase gene expression, as reported for the HIV pseudovirus neutralization assay (Chen et al., 2018). The 50% inhibitory dilution (EC50) was defined as the rhACE2 concentration at which the relative light units (RLUs) were reduced by 50% compared with the no treatment control wells. Briefly, the 3-fold diluted pseudovirus was incubated with naked or rhACE2 liposomes at 37° C. for 1 h before addition to the ACE2 and TMPRSS2 expressing 293T cells. Luciferase activity was measured using the Luciferase Assay System at 48 h following the manufacturer's instructions. The mixture of pseudovirus and liposomes was also imaged after incubation using TEM with negative staining (1% buffered phosphotungstic acid).

TEM Imaging. The pseudoviral particles suspended in cell culture medium or PBS were absorbed onto the carbon film of a TEM grid (Product #01810, TED PELLA, Redding, CA) by placing the grid carbon side down on top of a drop of sample for 20 min. The excess fluid was then blotted off using filter paper and the grid was placed within a large drop of PBS containing 2% paraformaldehyde for 5 min to fix the proteins of the pseudovirus. The excess fixing fluid was blotted off and rinsed with two drops of DI water. The sample was then stained with 2% phosphotungstic acid (PTA) for 30 sec. The excess staining fluid was blotted off and the grid was left to dry at room temperature overnight before being imaged by TEM.

### Example 2—Results

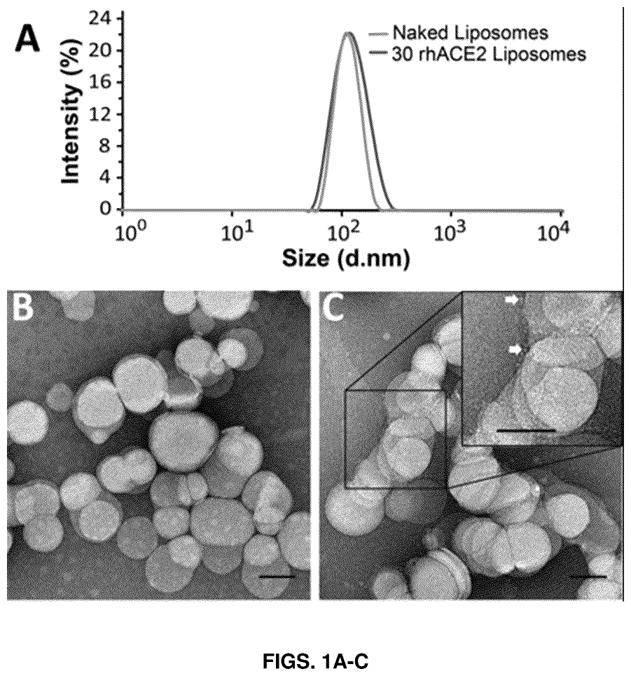

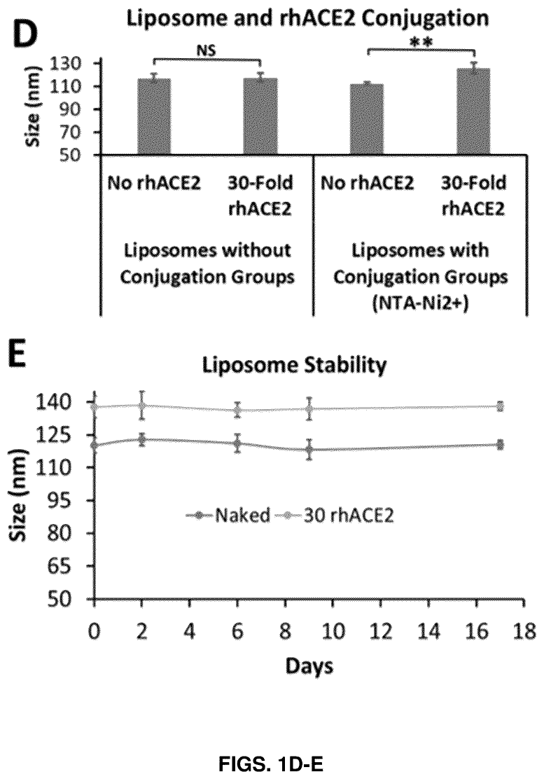

Synthesis and Characterization of rhACE2-Conjugated Liposomes. To modify ACE2 onto the liposome surface, the inventors included a lipid with one end group of nitrilotriacetic acid-Nickel (II) (NTA-Ni²⁺) in the liposome formulation and incubated preformed liposomes with his-tagged rhACE2 for conjugation based on the chelation chemistry between NTA-Ni²⁺ and his-tag (Mitchell et al., 2014; Jyotsana et al., 2019). The average hydrodynamic size of naked and rhACE2 liposomes was measured by dynamic light scattering (DLS) to be ˜112 to ˜125 nm respectively (FIG. 1A). The size increase after conjugation indicates that the protein was associated with liposomes after incubation. TEM with negative staining also revealed tiny spikes of less than 5 nm on the surface of rhACE2 liposomes in contrast to the smoother surface of naked liposomes (FIGS. 1B-C). However, the difference in surface morphology of liposomes alone may not be conclusive to support rhACE2 association with liposomes as the spikes could be artifacts caused by negative staining. To confirm the rhACE2/liposome association after the incubation for conjugation, the inventors included liposomes without NTA-Ni²⁺ lipid as a control in the conjugation experiment. No significant size change of NTA-Ni²⁺ absent liposomes from before to after the incubation was observed, which strongly supports that the size increase of the liposomes with NTA-Ni²⁺ was a convincing confirmation of rhACE2 association with liposomes (FIG. 1D). Both naked and rhACE2 liposomes showed high colloidal stability for more than two weeks when stored at 4° C., as found by monitoring their size distribution with DLS (FIG. 1E).

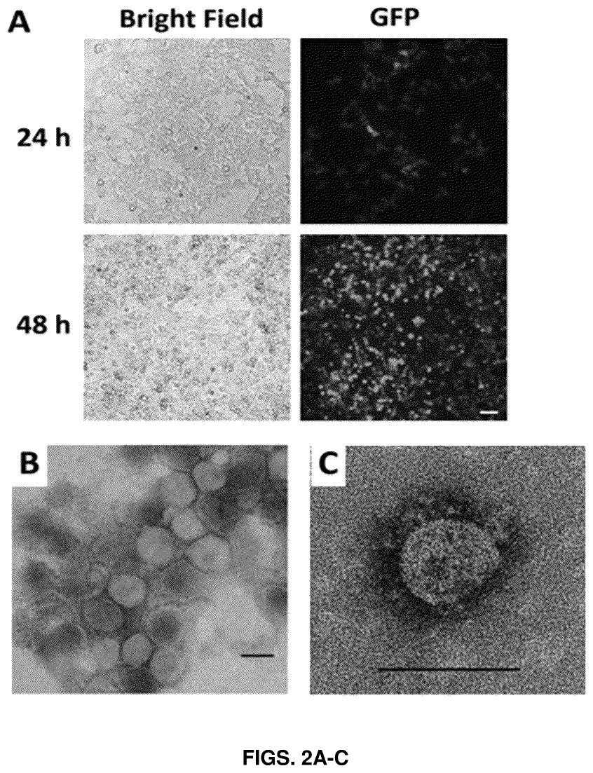

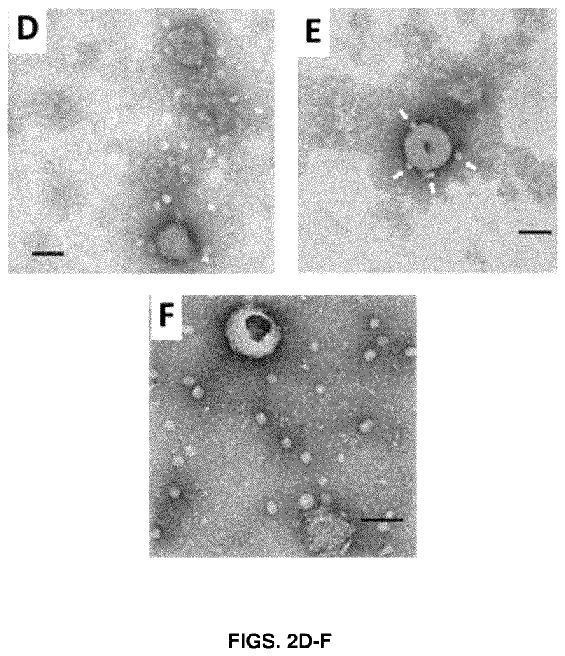

Construction and Characterization of SARS-CoV-2 Spike Pseudotyped Virus. Antiviral testing of highly infectious virus must be conducted in biosafety level (BSL) 3 or 4 facilities (Li et al., 2018). A popular alternative assay is to instead use pseudoviruses which are synthetic chimeras that consist of the cell entry-mediating surface proteins of the targeted virus and a surrogate viral core of a different virus (Chen & Zhang, 2021). Pseudoviruses are replication-incompetent as they lack the essential viral genes while possessing the same tropism and host entry pathway characteristics as live viruses, and thus are allowed to be safely handled in a BSL-2 lab (Cronin et al., 2005). The inventors constructed SARS-CoV-2 spike pseudotyped lentivirus and used it to test the SARS-CoV-2 neutralization potential of rhACE2 liposomes in 2.4. To prepare SARS-CoV-2 pseudovirus, 293T cells were transfected with a lentiviral packaging plasmid, a plasmid encoding the spike protein and a transfer plasmid encoding either GFP or luciferase. The success of pseudotyping inside cells was assessed by the expression of GFP (FIG. 2A). TEM with negative staining revealed an average size of ˜114.5±13.8 nm (n=200) for the pseudoviral particles collected from cell culture medium (FIG. 2B). Spike-like projections up to ˜20 nm on the surface can be clearly observed at higher magnifications, which is consistent with the size of trimeric spike proteins (FIG. 2C). The inventors attempted to concentrate the pseudoviruses using a polyethylene glycol (PEG) precipitation method in which PEG preferentially traps solvent and sterically excludes virions from the solvent phase. However, the inventors observed unexpected dissociation of spike proteins from the pseudovirus surface under TEM after the concentrated pseudoviral particles were redispersed in PBS (FIG. 2D). The spike proteins on the pseudovirus can be either partly or completely removed from the surface after the concentration step (FIGS. 2E-F). It is likely that the spike proteins of the pseudovirus are not as firmly anchored to the lentiviral membrane as those in SARS-CoV-2 live virus. A recent publication reported lentiviral particles pseudotyped with only the spike protein of SARS-CoV-2 are less infectious to ACE2 expressing cells than those pseudotyped also with the membrane and envelop protein in addition to the spike protein (Wang et al., 2021). The stabilization of spike proteins on the membrane of the pseudovirus by the membrane and envelope protein of SARS-CoV-2 may explain the increased infectivity.

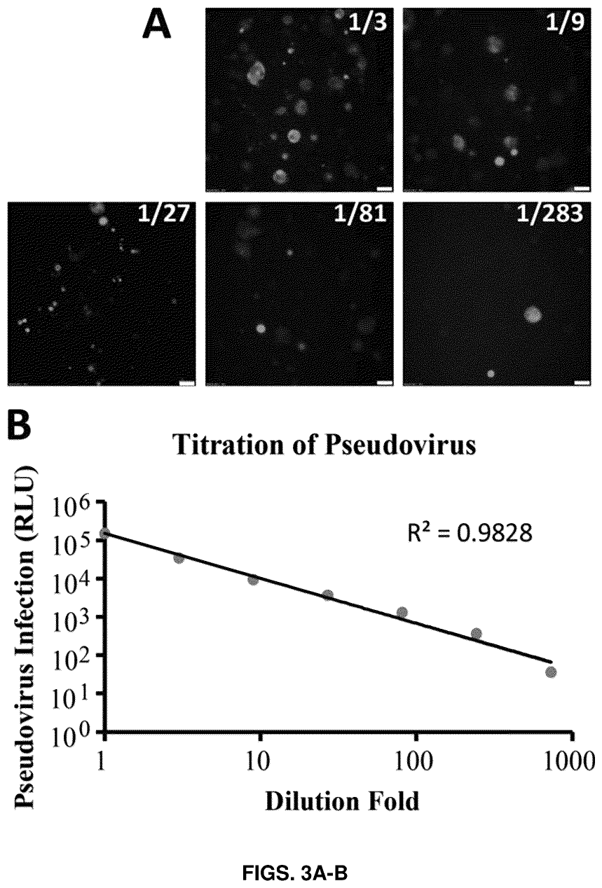

Infectivity of SARS-CoV-2 Pseudovirus. The inventors used 293T cells that stably express ACE2 and TMPRSS2 to test the infectivity titer of the pseudovirus (Neerukonda et al., 2021). The original medium containing the pseudovirus was sequentially diluted before addition to the cells. After 48 h, the infected cells, as indicated by the green fluorescence of pseudovirus labeled with GFP, was found to be inversely dependent on dilution factor (FIG. 3A). When luciferase-labeled pseudovirus was used, the infectivity was quantified with a luciferin assay (FIG. 3B). An excellent linear curve fit was established between the log scale of dilution factor and infectivity. Although the infectivity titer of this lentivirus-based pseudovirus might be lower than those with a vesicular stomatitis virus (VSV) backbone, their pseudotyping procedure is more straightforward and less time-consuming, and more importantly, they do provide a large dynamic range for generating neutralization dose-response curves (Fu et al., 2021).

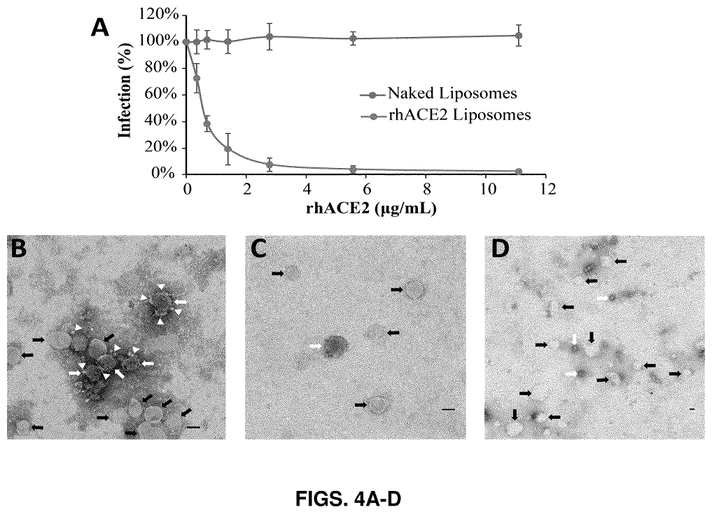

Neutralization of SARS-CoV-2 Pseudovirus by rhACE2 Liposomes. To test if rhACE2 liposomes can prevent the SARS-CoV-2 pseudovirus from infecting ACE2-TMPRSS2 expressing 293T cells, luciferase-labeled pseudovirus was incubated with the liposomes at specified concentrations of rhACE2 and 37° C. for 1 h before addition to the cell culture medium. This assay has been optimized, and is suitable for a variety of SARS-CoV-2 entry and neutralization screening assays (Neerukonda et al., 2021). Luciferase assay revealed that the rhACE2 liposomes potently inhibited the infectivity while the naked liposomes did not (FIG. 4A). The half-maximal inhibitory concentration (IC₅₀) was measured to be ˜0.41 ug/mL which is similar to other ACE2 presenting nanomaterials recently reported (El-Shennawy et al., 2020; Glasgow et al., 2020; Chen et al., 2021; Xie et al., 2021). To understand how the liposomes interacted with the pseudovirus, TEM with negative staining was performed to analyze the structure of pseudovirus after incubation with the liposomes. Based on their difference in contrast relative to background under TEM, liposomes and pseudoviruses can be visually differentiated in the mixture of the two. The spike proteins of the pseudoviruses could still be clearly observed after incubation with naked liposomes while not on the pseudovirus incubated with rhACE2 liposomes (FIGS. 4B-D). The spikes appeared to be removed by the rhACE2 liposomes during the incubation while the virus core was relatively structurally intact (FIGS. 4C-D). This is unexpected as we actually designed the liposomes conjugated with multiple rhACE2 to crosslink pseudoviruses coated with multiple spike proteins, assuming the spike proteins were firmly anchored on the pseudovirus membrane

### Example 3—Discussion

More than two years into the pandemic, SARS-CoV-2 mutations continue to emerge, impacting the effectiveness of once highly effective vaccines (Han & Ye, 2022). Only two drugs have been approved by the FDA for the treatment of COVID-19 with several others authorized for emergency use. Their effectiveness in reducing the morbidity and mortality associated with infection varies. One of the earliest treatments is transfusion of convalescent plasma from recovered patients (Sullivan & Roback, 2020). While its clinical benefits in some COVID-19 patients is encouraging, the treatment is limited by the availability of donor plasma. Five different antibodies have obtained emergency use authorization (EUA) but two of them have been paused due to lack of effectiveness (Lee et al., 2020). Of the three approved antivirals remdesivir, Paxlovid and molnupiravir, only Paxlovid has decent effectiveness but needs to be administered within five days of symptom onset and only for the treatment of mild-to-moderate disease (Wen et al., 2022). Repurposing clinical drugs haven't yielded effective drugs for COVID-19 although a significant amount of research efforts have been invested (Singh et al., 2020). Engineered nanomaterials that have been proposed for the treatment of COVID-19 include nanotraps which are rhACE2 coated polymer-lipid hybrid nanoparticles, ACE2 presenting nanodecoys or nanocatchers made by rupturing ACE2 overexpressing cells into nanoscale vesicles, and exosomes secreted from ACE2 expressing cells. While all these nanomaterials appear to be promising for COVID-19 treatment as they were demonstrated to potently inhibit viral entry, the complexity in their fabrication may impede further development towards the clinic (El-Shennawy et al., 2020; Chen et al., 2021; Xie et al., 2021; Rao et al., 2020; Zhang et al., 2021). There is still a pressing need to develop antivirals not only for the ongoing pandemic but also for future viral outbreaks that are likely to occur.

In order to demonstrate the potential of nanotechnology for broad-spectrum antiviral development, the inventors coated rhACE2 on nanoscale liposomes, arguably the most successful nanomaterial for drug delivery. rhACE2 liposomes are biocompatible and straightforward to fabricate in large scale with high repeatability (Akbarzadeh et al., 2013). rhACE2 proteins have been proposed for the treatment of COVID-19, but the small proteins may be rapidly cleared from the blood following administration (Sandhu & Kaur, 2020; Pang et al., 2020). The plasma circulating time of rhACE2 could be significantly extended when associated with liposomes as observed in many other nanomedicines (Allenm, 1997).

Liposomes have been used as a model of plasma membranes to study cellular entry and membrane fusion between viruses and cells or cellular organelles (Ott & Wunderli-Allenspach, 1994). While the inventors did not observe membrane fusion between the rhACE2 liposomes and pseudotyped SARS-CoV-2, it may occur under conditions more relevant to intracellular fluid such as low pH in lysosomes, or when SARS-CoV-2 live virus is used instead. Moreover, antiviral therapeutics may be encapsulated into the liposomes for direct delivery to individual virus (Lian & Ho, 2001). Membrane fusion between liposomes and viruses could bring the encapsulated antiviral therapeutics into direct contact with viral genes for degradation in a highly specific manner. The rhACE2 liposomes did not crosslink SARS-CoV-2 pseudovirus as expected. However, it is likely that the spikes are more firmly anchored in SARS-CoV-2 live viruses. In that case, the liposomes may crosslink the viruses into aggregates to prevent viral entry by sequestration and promote immune clearance due to increased size.

To conclude, the inventors developed rhACE2-conjugated liposomes for neutralization of SARS-CoV-2. The liposomes potently inhibited viral entry of pseudotyped SARS-CoV-2 into host cells by extracting the spike proteins from the pseudovirus surface. Receptor-coated nanoscale liposomes represent a new strategy for rapid antiviral development in the early stages of a viral outbreak. This strategy could be applied to target other viruses that require specific cell receptors for viral entry since the receptor proteins on the liposome surface can be readily replace. This broad-spectrum antiviral approach that can provide engineered liposomes in a timely manner for testing in preclinical and clinical studies in an expanding pandemic. One would thus be able to delay spread, attenuate virus evolution, and narrow the window between emergence and prevention and intervention.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VI. REFERENCES

The following references, to the extent that they provide exemplary procedural or other details supplementary to those set forth herein, are specifically incorporated herein by reference.
U.S. Pat. No. 4,728,578
U.S. Pat. No. 4,728,575
U.S. Pat. No. 4,737,323
U.S. Pat. No. 4,533,254
U.S. Pat. No. 4,162,282
U.S. Pat. No. 4,310,505
U.S. Pat. No. 4,921,706
International Application PCT/US85/01161
International Application PCT/US89/05040;
U.K. Patent Application GB 2193095
Adamson et al., Chemical Society Reviews 2021, 50, 3647.
Akbarzadeh et al., Nanoscale Res Lett 2013, 8, 102.
Allen, Drugs 1997, 54, 8.
Alijotas-Reig et al., Autoimmun Rev 2020, 19, 102569.
Baker et al., Nature Reviews Microbiology 2021, 20, 193.
Ballou et al., 2004.
Bangham et al., 1965.
Barnes et al., Nature 2020, 588, 682.
Chakravarty & Vora, Drug delivery and translational research 2021, 11, 748.
Chen & Zhang, Int J Biol Sci 2021, 17, 1574.
Chen et al., Human vaccines & immunotherapeutics 2018, 14, 199.
Chen et al., Matter 2021, 4, 2059;
Cheng et al., 1987.
Clercq & Li, Clinical Microbiology Reviews 2016, 29, 695.
Cohen, Biophysical journal 2016, 110, 1028.
Cronin et al., Current gene therapy 2005, 5, 387.
Deamer & Uster, 1983.
Dwarakanath et al., 2004.
Dubertret et al., 2002.
El-Shennawy et al., bioRxiv 2020, 2020.12.03.407031.
Farhood et al., 1995.
Frangioni, 2003.
Fu et al., Mol Ther Methods Clin Dev 2021, 20, 350.
Glasgow et al., Proceedings of the National Academy of Sciences 2020, 117, 28046.
Gregoriadis, 1979.
Han, & Ye, Journal of Medical Virology 2022, 94, 1366.
Hoffmann et al., Cell 2020, 181, 271.
Hope et al., 1985.
Jyotsana et al., Science Advances 2019, 5, eaaw4197.
Lee et al., Nature Microbiology 2020, 5, 1185.
Li et al., 2018, 28, e1963.
Li et al., Annual review of medicine 2022, 73, 1.
Lian & Ho, Journal of Pharmaceutical Sciences 2001, 90, 667.
Liposome Technology, 1984.
Mayer et al., 1986.
Mayhew et al., 1984.
Mayhew et al., 1987.
Meganck & Baric, Nature Medicine 2021, 27, 401.
Monteil et al., Cell 2020, 181, 905
Michalet et al., 2005.
Mitchell et al., Proceedings of the National Academy of Sciences 2014, 111, 930.
Neerukonda et al., PloS one 2021, 16, e0248348.
Ott & Wunderli-Allenspach, European Journal of Pharmaceutical Sciences 1994, 1, 323.
Pang et al., Acta Pharmacologica Sinica 2020, 41, 1255.
Patra et al., Journal of nanobiotechnology 2018, 16, 71.
Pardi & Weissman, Nature Biomedical Engineering 2020, 4, 1128.
Pellett et al., Handb Clin Neurol 2014, 123, 45.
Rao et al., Proceedings of the National Academy of Sciences 2020, 117, 27141;
Ryu, Molecular Virology of Human Pathogenic Viruses 2017, 31.
Sandhu & Kaur, Authorea Preprints 2020.
Shi et al., Nature Reviews Cancer 2017, 17, 20.
Singh et al., Pharmacological Reports 2020, 72, 1479.
Stayton et al., 2000.
Sullivan & Roback, Transfus Med Rev 2020, 34, 145.
Szoka & Papahadjopoulos, 1978.
Tang et al., Nano Today 2021, 36, 101019.
Wang et al., Int J Mol Sci 2021, 22.
Watanabe et al., Biochimica et Biophysica Acta (BBA)—General Subjects 2019, 1863, 1480.
Wen et al., Annals of Medicine 2022, 54, 516.
West, 2004.
Xie et al., Advanced Materials 2021, 33, 2103471.
Zhang et al., Proceedings of the National Academy of Sciences 2021, 118, e2102957118.

1. A method of neutralizing an enveloped virus comprising:
a) providing a nanoparticle comprising a surface exposed cell receptor molecule that mediates entry of an enveloped virus into host cells; and
b) administering the nanoparticle to an individual having or at risk of infection with the enveloped virus, wherein the cell receptor mediates a specific interaction event between the nanoparticle and the enveloped virus.

2. The method of claim 1, wherein the cell receptor molecule is any one of the cell receptors or any combination of two or more cell receptors from the following list: selectin a-Dystroglycan, transferrin, Histo-blood group antigen (HBGA) receptors, heat-shock protein 70 (HSP70), sialic acid, ephrin B2, DC-SIGN, CD4, CD21, CD81, CD155, ICAM-1, low-density lipoprotein receptor (LDLR), lactoseries tetrasaccharide c (LSTc), GM1, JAM, laminin receptor, ACE-2, L-SIGN, neclin-1/2, HVEM, nectin-4, human scavenger receptor class B, member 2 (SCARB2), P-selectin glycoprotein ligand-1 (PSGL 1), glucose transporter type 1 (GLUT-1), neuropilin-1, coxsackievirus adenovirus receptor (CAR), av integrins, T-cell immunoglobulin and mucin domain 1 (TIM-1), human Niemann-Pick C1 (NPC1), scavenger receptor class B type I (SR-B1), major histocompatibility complex class II (MHC-11), decay-accelerating factor (OAF), CAR (occluding), integrins, and/or C-C chemokine receptor type 5 (CCR5).

3. The method of claim 1, wherein the enveloped virus is coronavirus (CoV), such as SARS-CoV-2, Norovirus, Japanese encephalitis virus, Influenza A, Henipahvirus, Bunyavirus, Hepatitis A virus, Poliovirus, Rhinovirus (major group), Rhinovirus (minor group), John Cunningham polyomavirus, SV40 polyomavirus, Reovirus, Sindbis virus, Herpes simplex virus 1/2, Measles virus, Enterovirus 71, Human T cell leukemia virus 1, Adenovirus 2, Ebola virus, hepatitis C virus, Epstein-Barr virus, Rotavirus, or HIV.

4. The method of claim 1, wherein nanoparticle further comprises a DNA or RNA degrading reagent, such as any one or any combination from the following list: DNase I, DNase II, micrococcal nuclease, RNase A, RNase D, RNase E, RNase G, RNase H, RNase L, RNase P, RNase PH, RNase PhyM, RNase R, RNase T, RNase T1, RNase T2, RNase V, RNase U2, Oligoribonuclease, Exoribonuclease I, and/or Exoribonuclease II.

5. The method of claim 1, wherein the nanoparticle is a liposome.

6. The method of claim 5, wherein the liposomes are unilamellar liposomes, wherein majority of the liposomes have a size from 30 to 500 nm.

7. The method of claim 1, wherein administration comprises systemic administration, regional administration, or administration local to a site of infection, such as intravenous, intra-arterials, intranasal, inhalation, oral, parenteral, vaginal, or rectal.

8. The method of claim 1, wherein the nanoparticles are administered into the circulation of an individual by intravenous administration.

9. The method of claim 1, wherein the nanoparticles are administered into the lymphatic circulation of the individual.

10. The method of claim 1, further comprising treating the individual with a second anti-viral therapy.

11. A nanoparticle comprising a surface exposed cell receptor molecule that mediates entry of an enveloped virus into host cells.

12. The nanoparticle of claim 11, wherein the cell receptor molecule is any one or any combination of two or more cell receptors from the following list: selectin a-Dystroglycan, transferrin, Histo-blood group antigen (HBGA) receptors, heat-shock protein 70 (HSP70), sialic acid, ephrin B2, DC-SIGN, CD4, CD21, CD81, CD155, ICAM-1, low-density lipoprotein receptor (LDLR), lactoseries tetrasaccharide c (LSTc), GM1, JAM, laminin receptor, ACE-2, L-SIGN, neclin-1/2, HVEM, nectin-4, human scavenger receptor class B, member 2 (SCARB2), P-selectin glycoprotein ligand-1 (PSGL 1), glucose transporter type 1 (GLUT-1), neuropilin-1, coxsackievirus adenovirus receptor (CAR), av integrins, T-cell immunoglobulin and mucin domain 1 (TIM-1), human Niemann-Pick C1 (NPC1), scavenger receptor class B type I (SR-B1), major histocompatibility complex class II (MHC-11), decay-accelerating factor (OAF), CAR (occluding), integrins, and/or C-C chemokine receptor type 5 (CCR5).

13. The nanoparticle of claim 11, wherein nanoparticle further comprises a DNA or RNA degrading reagent, such as any one of any combination from the following list: DNase I, DNase II, micrococcal nuclease, RNase A, RNase D, RNase E, RNase G, RNase H, RNase L, RNase P, RNase PH, RNase PhyM, RNase R, RNase T, RNase T1, RNase T2, RNase V, RNase U2, Oligoribonuclease, Exoribonuclease I, and/or Exoribonuclease II.

14. The nanoparticle of claim 11, wherein the nanoparticle is a liposome.

15. The nanoparticle of claim 14, wherein the liposomes are unilamellar liposomes, wherein majority of the liposomes have a size from 30 to 250 nm.

16. A kit comprising a nanoparticle comprising a surface exposed cell receptor molecule that mediates entry of an enveloped virus into host cells.

17. The kit of claim 16, wherein the cell receptor molecule is any one or any combination of two or more cell receptors from the following list: selectin a-Dystroglycan, transferrin, Histo-blood group antigen (HBGA) receptors, heat-shock protein 70 (HSP70), sialic acid, ephrin B2, DC-SIGN, CD4, CD21, CD81, CD155, ICAM-1, low-density lipoprotein receptor (LDLR), lactoseries tetrasaccharide c (LSTc), GM1, JAM, laminin receptor, ACE-2, L-SIGN, neclin-1/2, HVEM, nectin-4, human scavenger receptor class B, member 2 (SCARB2), P-selectin glycoprotein ligand-1 (PSGL 1), glucose transporter type 1 (GLUT-1), neuropilin-1, coxsackievirus adenovirus receptor (CAR), av integrins, T-cell immunoglobulin and mucin domain 1 (TIM-1), human Niemann-Pick C1 (NPC1), scavenger receptor class B type I (SR-B1), major histocompatibility complex class II (MHC-11), decay-accelerating factor (OAF), CAR (occluding), integrins, and/or C-C chemokine receptor type 5 (CCR5).

18. The kit of claim 16, wherein nanoparticle further comprises a DNA or RNA degrading reagent, such as any one of any combination from the following list: DNase I, DNase II, micrococcal nuclease, RNase A, RNase D, RNase E, RNase G, RNase H, RNase L, RNase P, RNase PH, RNase PhyM, RNase R, RNase T, RNase T1, RNase T2, RNase V, RNase U2, Oligoribonuclease, Exoribonuclease I, and/or Exoribonuclease II.

19. The kit of claim 16, wherein the nanoparticle is a liposome.

20. The kit of claim 19, wherein the liposomes are unilamellar liposomes, wherein majority of the liposomes have a size from 30 to 250 nm.
